(19) United States
(12) Patent Application Publication
(10) Pub. No.: US 2022/0120725 A1
(43) Pub. Date: Apr. 21, 2022

(54) AFLATOXIN CONTAMINATION RISK WARNING MOLECULE AND USE THEREOF

(71) Applicant: OIL CROPS RESEARCH INSTITUTE, CHINESE ACADEMY OF AGRICULTURAL SCIENCES, HUBEI (CN)

(72) Inventors: Peiwu LI, Hubei (CN); Qi ZHANG, Hubei (CN); Huali XIE, Hubei (CN); Xiupin WANG, Hubei (CN); Xiaofeng YUE, Hubei (CN); Yizhen BAI, Hubei (CN)

(73) Assignee: OIL CROPS RESEARCH INSTITUTE, CHINESE ACADEMY OF AGRICULTURAL SCIENCES, HUBEI (CN)

(21) Appl. No.: 17/502,064

(22) Filed: Oct. 15, 2021

(30) Foreign Application Priority Data
Oct. 15, 2020 (CN) 202011106439.4

(51) Int. Cl. G01N 33/02; G01N 30/06; G01N 30/72; G01N 33/24

(52) U.S. Cl. CPC G01N 33/02; G01N 30/06; G01N 2030/027; G01N 33/24; G01N 30/72

(57) ABSTRACT

The present invention relates to an aflatoxin contamination risk warning molecule and use thereof. The steps are as follows: weighing a quantitative sample, extracting the aflatoxin contamination risk warning molecule to obtain a sample extract, and detecting and analyzing the sample extract to obtain a quantitative result of the aflatoxin contamination risk warning molecule; performing modeling with a chemometrics method using the content of one or more of the aflatoxin contamination risk warning molecules as a variable to obtain a classification prediction model, and performing risk assessment on aflatoxin contamination risk of the sample based on the classification prediction model, wherein a warning molecule of an aflatoxin toxigenic strain is one or a combination of more than one of versiconol (VOH), versicolorin B (Ver B), and 5-methoxysterigmatocystin (5-MST).

### CROSS-REFERENCE TO RELATED APPLICATION

This application claims the priority benefit of China application serial no. 202011106439.4, filed on Oct. 15, 2020. The entirety of the above-mentioned patent application is hereby incorporated by reference herein and made a part of this specification.

### BACKGROUND

### Technical Field

The present disclosure relates to an assessment method for aflatoxin contamination risk, and belongs to the field of analysis and detection.

### Description of Related Art

Mycotoxins are secondary metabolites generated from filamentous fungi, and may, in an entire industrial chain, contaminate peanuts, corn, cotton, nuts and other crops. They have a high incidence worldwide, causing huge economic losses due to mycotoxin contamination of 25% of crops each year in the world, while seriously endangering people's lives and health. For example, they have carcinogenic, immunosuppressive, hepatotoxic, nephrotoxic and neurotoxic properties, and the like. Aflatoxin is mainly derived from Aspergillus flavus, and is considered to be one of the ten most horrible fungi in the world. This fungus is widely distributed in the world, including China, and is a major causative factor of regional liver cancer in China. Currently, many countries have established a strict aflatoxin limit standard, so as to ensure the quality and safety of agricultural products and avoid the resulting trade barriers. It is obvious that the development of an early aflatoxin warning method becomes urgent.

The contamination risk of aflatoxin is mainly divided into contamination by toxigenic Aspergillus flavus and contamination by aflatoxin.

In the present disclosure, in order to realize risk warning of aflatoxin contamination, two strategies are provided: (1) a toxigenic Aspergillus flavus bio-warning molecule is developed to early identify the toxigenicity of Aspergillus flavus in peanut or soil so as to early warn aflatoxin contamination; and (2) the severity of aflatoxin contamination is predicted by dynamically monitoring the toxigenicity-related warning molecules. It is well known that fungi have evolved thousands of secondary metabolites as chemical weapons or armor to occupy favorable ecological niches and protect their food from competitors. Theoretically, these diversities pave the way for screening of a bio-warning molecule of toxigenic Aspergillus flavus at the subspecies level in this study. Here, provided is that the metabolic diversity properties of the Aspergillus flavus population may be systematically investigated in combination with machine learning techniques to screen for warning molecules that may effectively distinguish high-toxigenicity and low-toxigenicity strains, and to predict the severity of aflatoxin contamination in agricultural products based on the dynamics of the warning molecules.

### SUMMARY

The technical problem to be solved by the present disclosure is to provide an aflatoxin contamination risk warning molecule and use thereof in view of the lack of existing early warning methods before the occurrence of aflatoxin contamination.

In order to solve the above technical problem, the technical solution adopted by the present disclosure is as follows:

use of an aflatoxin contamination risk warning molecule in aflatoxin contamination risk warning is provided, wherein the aflatoxin contamination risk warning molecule is one or a combination of more than one of versiconol (VOH), versicolorin B (Ver B) and 5-methoxysterigmatocystin (5-MST). The use is to warn the aflatoxin contamination risk based on the presence or absence or content of one or more of the above warning molecules.

A method for warning aflatoxin contamination risk based on the aflatoxin contamination risk warning molecules described above includes the following steps:

weighing a quantitative sample, extracting the aflatoxin contamination risk warning molecule to obtain a sample extract, and detecting and analyzing the sample extract to obtain a quantitative result of the aflatoxin contamination risk warning molecule;

performing modeling with a chemometrics method by using the content of one or more of the aflatoxin contamination risk warning molecules described above as a variable to obtain a classification prediction model, inputting the quantitative result of the aflatoxin contamination risk warning molecule, and outputting a risk assessment result based on the classification prediction model to warn aflatoxin contamination of the sample.

According to the above solution, the chemometrics method is a multivariate statistical analysis method such as hierarchical cluster analysis, least partial square orthogonal projection, and random forest.

According to the above solution, after the sample is cultured for 3-4 days, the sample is taken to detect a warning molecule of toxigenic Aspergillus flavus, and a quantitative value of the warning molecule is directly input to the classification prediction model to predict the aflatoxin contamination risk.

According to the above solution, after the sample is cultured for 3-4 days, the sample is taken to detect a warning molecule of toxigenic Aspergillus flavus, and if the content of 5-methoxysterigmatocystin is greater than a threshold value of 34.7 μg/kg, whether the content of VerB is greater than 96.35 μg/kg is further used to determine the contamination risk of the sample. If the content of VerB is greater than 96.35 μg/kg, the sample is a high-risk aflatoxin contamination sample; and if the content of VerB is less than or equal to 96.35 μg/kg, the sample is a medium-risk aflatoxin contamination sample. The medium-risk sample may be further input into the accurate classification prediction model for validation.

According to the above solution, the above method further includes screening a suspected sample, pre-treating the screened suspected sample, detecting the warning molecule of toxigenic Aspergillus, and outputting the risk assessment result based on the classification prediction model to conduct early warning assessment of the risk of aflatoxin contamination of the sample, specifically: detecting the aflatoxin content of the sample; subjecting a sample in which aflatoxin is not detected or the aflatoxin content does not exceed the standard to an accelerated microbial metabolism culture experiment (that is, added to a sterile culture dish containing a mold medium, placed in a constant temperature incubator to be incubated for 3-4 days), wherein Aspergillus will grow in the suspected contaminated sample; quenching the suspected sample with liquid nitrogen and grinding for later use; and detecting the aflatoxin content of the sample, wherein a sample with the aflatoxin content higher than a national limit standard is directly identified as a high-risk sample, i.e., the suspected sample.

According to the above solution, the sample is an agricultural product or food, including peanuts and so on.

According to the above solution, extracting the aflatoxin contamination risk warning molecule includes: performing first extraction by using a solution with a volume ratio of methanol to acetonitrile to water being 2-4:2-4:0-1, and then performing second extraction by using another extraction solution (a volume ratio of methanol to dichloromethane to ethyl acetate being 1-3:1-2:1-2) to extract the aflatoxin contamination risk warning molecule, and then centrifugating at a high speed to obtain the sample extract.

According to the above solution, analyzing the sample includes: subjecting the sample to detection and analysis by liquid chromatography-high resolution mass spectrometer, in which a chromatographic column is a C₁₈ reverse chromatographic column, and a mass spectrometry acquisition mode is divided into a positive ion mode and a negative ion mode which are operated separately, the acquisition mode is data-dependent acquisition mode, and primary mass spectrometry data and secondary fragment ion data are simultaneously acquired to perform qualitative and quantitative analysis, thereby obtaining the analysis results of the warning molecule.

According to the above solution, the detection and analysis by the liquid chromatography-high resolution mass spectrometer contains an internal standard substance, the internal standard being camphoric acid (the negative ion mode) and 2-chlorophenylalanine (the positive ion mode).

According to the above solution, the qualitative analysis of the warning molecule includes: determining mass deviation within 5 ppm according to the precise mass number of the primary mass spectrometry of the warning molecule, and then comparing the main characteristic ion peaks of the secondary mass spectrometry in combination with the secondary mass spectrogram to perform the qualitative analysis; and the quantitative analysis includes: in combination with the internal standard substance, performing the quantitative analysis based on a pre-established standard curve of the chromatographic peak area of each warning molecule/the peak area of the internal standard substance-warning molecule concentration. The mass spectra of the warning molecules are shown in FIG. 11(a)-(c).

The main characteristic ion peaks of the secondary mass spectrometry for the warning molecule 5-methoxysterigmatocystin (5-MST) include: 350, 0809 Da, 340.0571 Da, 322.04675 Da, 311.05469 Da and 285, 0098 Da.

The characteristic ion peaks of the secondary mass spectrometry for the warning molecule versiconol (VOH) include: 329.06546 Da, 341.09506 Da, and 359.07596 Da.

The characteristic ion peaks of the secondary mass spectrometry for the warning molecule versicolorin B (Ver B) include: 311.0542 Da, 311.0187 Da, and 283.0238 Da.

The standard curves of the chromatographic peak area of each warning molecule/the peak area of the internal standard substance-warning molecule concentration are respectively as follows:

5-Methoxysterigmatocystin: Y=317.3X−114190.2;

Versicolorin_B: Y=232.9X−142191.5;

Versiconol: Y=62.5X+39562.3,

wherein X is warning molecule concentration ‘Y is the chromatographic peak/the peak area of the internal standard.

According to the present disclosure, a machine learning algorithm, such as least partial square orthogonal projection, and random forest, is used to train a training set containing 334 samples so as to screen highly stable warning molecules of middle- or high-toxigenic strains with large differences between high- and low-toxigenic strains, including 5-methoxysterigmatocystin, versiconol, and versicolorin_B, and then the remaining 234 samples are used as an independent validation set to validate the warning molecules by using the machine learning algorithm such as least partial square orthogonal projection and random forest. The validation results show that the top warning molecules screened by the validation set have the same selection results as those of the training set, including 5-methoxysterigmatocystin, versiconol, and versicolorin_B. Further, we have developed a simple and intuitive decision rule by using an interpretable machine learning model, as shown in FIG. 10. When the warning molecules are detected, the two warning molecule thresholds trained by machine learning may be used to quickly identify the future contamination risk level of the sample, thereby guiding the classification decision.

The screening of the warning molecule for toxigenic strains of aflatoxin described above specifically includes: the collected 568 samples are firstly divided into a training set of 334 samples, and an independent validation set of 234 samples. The training set containing 334 samples is trained by using a machine learning algorithm such as least partial square orthogonal projection and random forest, to screen highly stable warning molecules of middle- to high-toxigenic strains, for example, BioM8 (5-Methoxysterigmatocystin), BioM-18 (Versiconol), and BioM-36 (Versicolorin_B), as shown in FIG. 4, wherein the molecular formula, accurate mass number, and other information of these molecules are shown in Table 1. The remaining 258 samples are then used as the independent validation set to validate the warning molecules. The validation result shows that these warning molecules are also screened in the independent validation set and ranked in the top (as shown in FIG. 5). The model constructed by these warning molecules has the best prediction accuracy (as shown in FIG. 5). Therefore, these warning molecules are selected to construct warning molecules for the toxigenic Aspergillus flavus or strains, or the combination thereof, for the assessment of aflatoxin contamination.

TABLE 1
LC-HRMS information of aflatoxin B1 and bio-warning molecules

| | | | Precise mass numbers | | Mass |
|---|---|---|---|---|---|
| | Molecular | | Theoretical | Experimental | deviation |
| | Formula | Ionic mode | value | value | (ppm) |
| Aflatoxin B₁ | C₁₇H₁₂O₆ | [M + H]⁺ | 313.07066 | 313.07012 | 1.73 |
| 5-Methoxysterigmatocystin | C₁₉H₁₄O₇ | [M + H]⁺ | 355.08122 | 355.08068 | 1.52 |
| Versicolorin_B | C₁₈H₁₂O₇ | [M − H]⁻ | 339.05102 | 339.05096 | 0.17 |
| Versiconol | C₁₈H₁₆O₈ | [M − H]⁻ | 359.07724 | 359.07733 | 0.25 |

The beneficial effects of the present disclosure are as follows.

1. According to the present disclosure, the metabolic diversity of the Aspergillus flavus population in China is systematically evaluated for the first time, and an advanced machine learning data analysis method is used to screen warning molecules for toxigenic Aspergillus flavus for the first time. An accurate warning molecule is provided for the identification of toxigenic fungi at the subspecies level, and an original warning molecule is provided for the early warning of mycotoxins. Meanwhile, the strategies of this study can draw inferences about other cases from one instance, and can be extended to all other microbial subspecies, providing a methodological reference for accurate identification and classification. A new way is provided to solve the problem that there is no early warning molecule to monitor in the field of food quality and safety research.

2. The population metabolomics screening of warning molecules used in the present disclosure provides an example for early warning of mycotoxin contamination.

3. According to the present disclosure, a machine learning method is further used to screen the warning molecules to study the difference of different machine learning algorithms, and a combination of robust warning molecules is obtained by comparing the stability of different screening results so that the classification model can achieve the highest classification accuracy under the condition of minimum detection of warning molecules.

4. The warning molecules found in the present method are original and can effectively achieve accurate identification of toxigenic Aspergillus flavus, and the established detection warning molecules are highly sensitive, thereby achieving high-sensitivity detection and analysis.

### BRIEF DESCRIPTION OF THE DRAWINGS

FIG. 1 is a flowchart of the sampling experimental design solution for screening the toxigenicity of Aspergillus flavus population strains in China, in which the sample is carefully selected according to the toxigenicity of strains, and geographical and ecological origin.

FIG. 2 shows the toxigenicity data and classification of non-toxigenic, low-toxigenic and medium- or high-toxigenic Aspergillus flavus population strains.

FIG. 3 shows regional division of the toxigenicity of Aspergillus flavus population strains in northern, central and southern regions of China.

FIG. 4 shows a graph of the results of variable importance screening.

FIG. 5 shows a graph of significant variables corroborated by an independent validation set and screened by a random forest model.

FIG. 6(a)-(c) show Pearson correlation analysis correlating toxigenicity of Aspergillus flavus with the warning molecule, FIG. 6(a) shows the correlation analysis between 5-methoxysterigmatocystin and toxigenicity, FIG. 6(b) shows the correlation analysis between versicolorin_B and toxigenicity, and FIG. 6(c) shows the correlation analysis between versiconol and toxigenicity.

FIG. 7 shows standard curves of aflatoxin B0 and 3 warning molecules for quantitative analysis of aflatoxin B1 and warning molecules.

FIG. 8(a) shows a workflow for early warning by monitoring the metabolic warning molecules of toxigenic Aspergillus fungi; FIG. 8(b) shows a heat map illustrating the effective differentiation of the warning molecules from 86 suspected samples.

FIG. 9(a)-(b) are a graph of a growth and decline rule of warning molecules related to the severity of contamination and aflatoxin biosynthesis.

FIG. 10 is a simple and intuitive operable decision using the warning molecules and their thresholds.

FIG. 11(a)-(c) show the secondary mass spectra and the multi-level mass spectral fragmentation ion tree of 3 warning molecules for the qualitative analysis and comparison of the warning molecules.

### DESCRIPTION OF THE EMBODIMENTS

The present disclosure will be further described in detail below in combination with specific embodiments, in order to enable those skilled in the art to better understand the technical solutions of the present disclosure.

The abbreviations of the compounds involved in the present disclosure: versiconol (VOH), versicolorin B (Ver B), aflatoxin B1 (AFB1), aflatoxin B2 (AFB2), and 5-methoxysterigmatocystin (5-MST).

In the following embodiments, a process for establishing a standard curve includes: 200 mg of Aspergillus flavus mycelia are weighed and added into a mortar and ground in liquid nitrogen, and then 5 mL of a PBS buffer solution is added. The resulting solution is gradiently diluted to 0.01, 0.05, 0.1, 0.5, 1, 2, 5, 10, and 100 μg/mg of mycelium liquid to construct the standard curve.

### Embodiment 1 Screening of Warning Molecules for Toxigenic Strains of Aflatoxin

The warning molecules that may effectively distinguish high-toxigenicity strains and low-toxigenicity strains were screened by systematically investigating the metabolic diversity properties of the Aspergillus flavus population, in combination with machine learning techniques, mainly including the following steps.

Selection of a representative sample: samples were prepared according to standard operating procedures. With reference to information of a strain bank of the Aspergillus flavus population, the strain was selected from the strain bank depending on geographical origin.

Sample preparation: Aspergillus flavus was activated on a solid medium and cultured by using an Sabouraud liquid medium that contributed to the production of toxins, to obtain mycelial samples of different Aspergillus flavus strains.

Sample pre-treatment: a metabolome sample was quenched, mycelia were ground, and an extraction solution containing an internal standard was added for extraction. The extract was centrifuged at a high speed, and filtered with a filter membrane to yield a sample to be uploaded to a machine.

Sample detection: the aforementioned sample was subjected to detection and analysis by liquid chromatography-high resolution mass spectrometry for qualitative and quantitative analysis, to obtain the analytical results of the warning molecules. Internal standard substances were used in detection by liquid chromatography-high resolution mass spectrometer, and the internal standard substances were camphoric acid (a negative ion mode) and 2-chlorophenylalanine (a positive ion mode).

Generally, the qualitative analysis described above includes first-level, second-level and third-level qualitative analysis results. The first-level qualitative result is a result that the detected compound is verified by the standards and has completely matched information in primary and secondary mass spectrometry and consistent retention time. The second-level qualitative result is a result of a qualitative compound that has a 50% or higher matching score between the characteristic peaks extracted from the sample and the secondary mass spectrometry information in the public database. The third-level qualitative result is a result of a sample that has a deviation less than 5 ppm from the first-order accurate mass number of the compounds already reported in the study species.

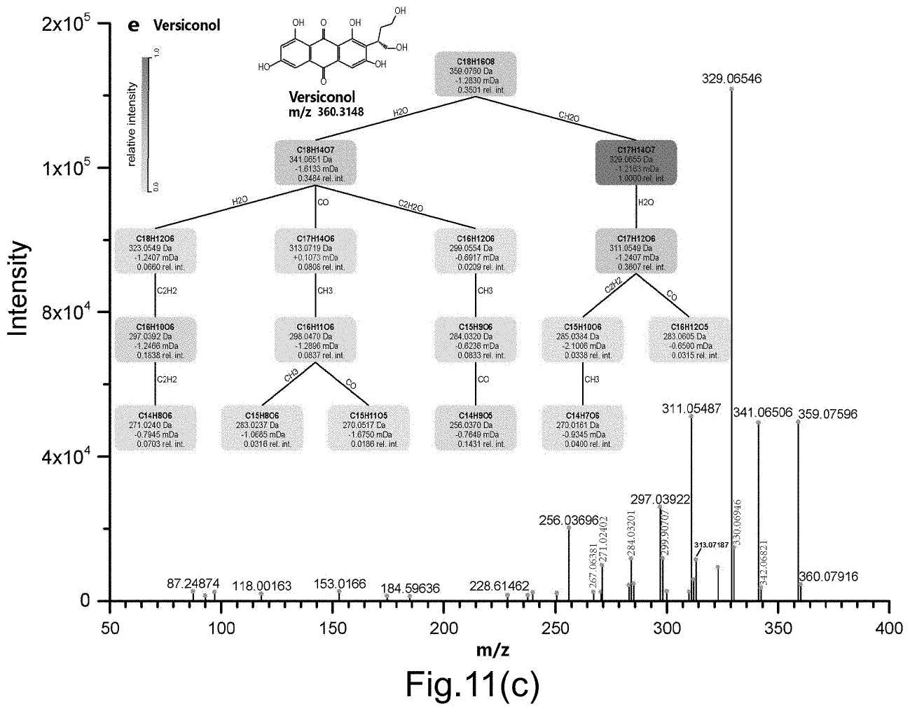

The qualitative analysis in the disclosure includes: determining the mass deviation to be within 5 ppm, based on the accurate mass number of the primary mass spectrometry of the warning molecule; comparing the secondary mass spectrogram with the main characteristic ion peaks of the secondary mass spectrogram of the warning molecule (as shown in FIG. 11(a)-(c)) for qualitative analysis; performing the quantitative analysis based on the pre-established standard curve of the chromatographic peak area/the peak area of the internal standard-warning molecule concentration, in combination with the internal standard substances.

After qualitatively obtaining the metabolite list, software Xcaliber 3.1 was used to extract and detect the peaks of the qualitative metabolites, to obtain a raw data list of the peaks. Metabonomic data preprocessing includes: the peaks were firstly extracted from raw data containing primary and secondary mass spectrometry information (which may be imported into Compound Discovery 2.1 for peak extraction from raw data), while the chemical molecular formula thereof was predicted, and then the peaks were aligned, and the accurate mass numbers in primary and secondary mass spectrometry were matched with the mass spectrometry database for qualitative analysis to obtain the raw data list of the peaks.

The specific steps are as follows.

(1) Experimental Design, Sample Pre-Treatment, and Detection and Identification of Metabolite

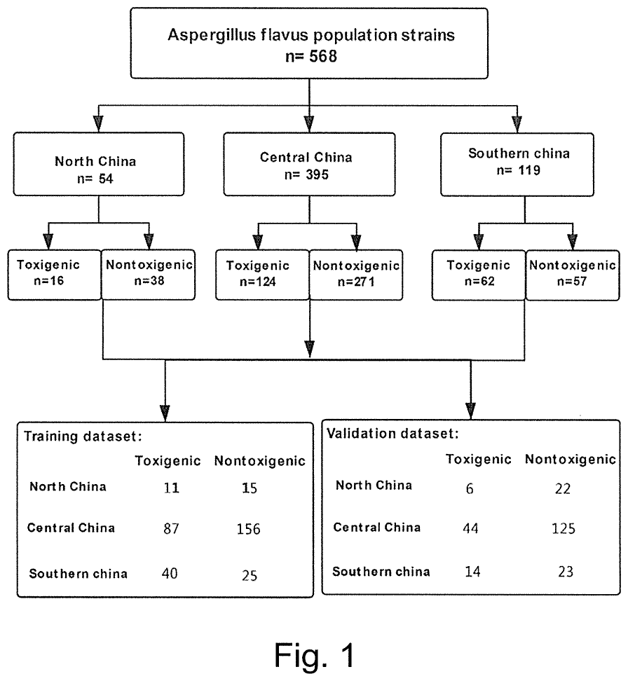

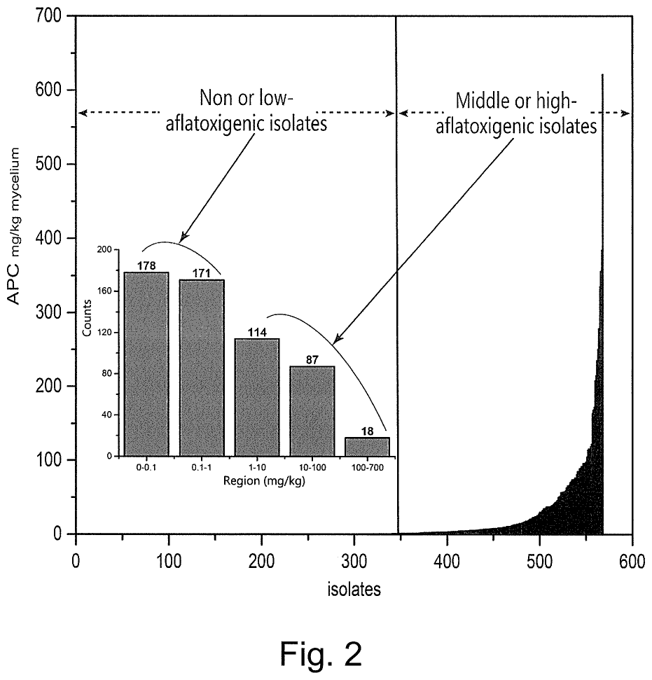

The representativeness of the sample is of vital importance, in order to screen a potential warning molecule of a toxigenic Aspergillus flavus strain to assess the risk of aflatoxin contamination. For this purpose, we carefully selected strains, as a sample, from a strain bank established from samples taken from 337 counties for this study, and the strains had different toxigenicity and were derived from the northern, central and southern regions. As shown in FIG. 1, the sampling experimental design solution for screening the toxigenicity of Aspergillus flavus population strains is as follows: according to the proportion of peanut cultivation and the ecogeographic type, we selected 68 strains from the northern region, wherein 33.8% of the strains were highly toxigenic and 66.2% of the strains were low- or non-toxigenic strains. From the central region, 413 strains were selected, wherein 42.6% of the strains were highly toxigenic strains and 57.4% of strains were low or non-toxigenic strains. From the southern region, 125 representative strains were selected, wherein 59.2% of the strains were highly toxigenic strains and 40.8% of the strains were low or non-toxigenic strains. Metabolomic data collection was performed on these 568 samples, and these strains from different sources were randomly distributed during sample preparation and data collection or subsequent marker screening, while meeting the requirements of the solution in FIG. 1. Among these, 334 samples were used as a training set to discover warning molecules and the remaining 234 samples were used as an independent validation set to assess the robustness of the screened warning molecule. The classification of non-toxigenic, low-toxigenic and middle or high-toxigenic Aspergillus flavus population strains is shown in FIG. 2. The rule for identification and classification of toxigenicity is: the strains were divided into five groups according to their toxigenicity values, in which a first group consisted of non-toxigenic strains with 0-0.1 mg/kg of mycelium, a second group consisted of low-toxigenic strains with 0.1-1 mg/kg of mycelium, a third group consisted of medium-toxigenic strains (1-10 mg/kg of mycelium), a fourth group consisted of medium or high-toxigenic strains (10-100 mg/kg of mycelium), and a fifth group consisted of high-toxigenic strains (100-700 mg/kg mycelium), as shown in FIG. 2.

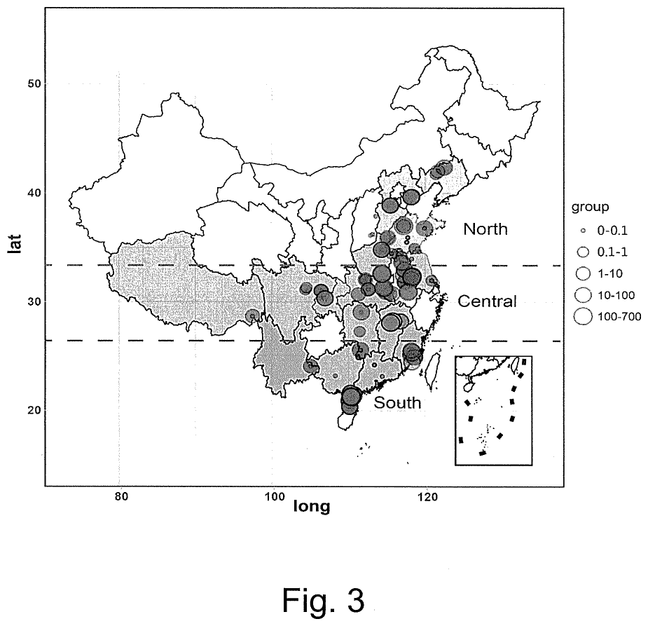

FIG. 3 shows the regions division of the toxigenicity of Aspergillus flavus population strains in the northern, central and southern regions of China.

(2) The Experimental Method of Strain Culture and Sample Pre-Treatment

A PDA agar medium (Becton, Dickinson and company, France) was inoculated with the Aspergillus flavus conidia and the Aspergillus flavus conidia were incubated at 29±1° C. for 8-10 days. Spores were washed by using 0.1% of Tween-80 to obtain a spore suspension. Spores were counted by using a hemocytometer plate in combination with a microscope and the concentration of the spore suspension was calculated. A liquid medium with 0.25% of a yeast extract, 0.1% of K₂HPO₄, 0.05% of MgSO₄-7H₂O, and 10% of glucose, was prepared, and the pH of the medium was adjusted to 6.0. Then, 50 mL of the prepared liquid medium was subpackaged in a triangular culture flask and sterilized at high temperature for 20 min. The sterilized liquid medium was inoculated with the spores at 5×10⁵ spores/mL, and the spores were incubated in a shaker at 180 rpm at 29±1° C. for 5 days, and filtered to collect the obtained mycelial sample.

Quenching and pre-treatment of the sample: after obtaining the mycelial sample as described above, the mycelial sample was quickly filtered, rinsed with 10 mL of saline (0.9% (wt/vol) NaCl) at 4° C., and then quenched by using liquid nitrogen. The sample was stored in a freezer at −80° C. for drying, and then lyophilized by using a freeze dryer. 50 mg of the sample was weighed, and was subjected to extraction with 1 ml of an extraction solution containing an internal standard substance (methanol:acetonitrile:water=2:2:1), 5 steel beads were added, and then the sample was triturated by using a homogenizer. The sample was subjected to ultrasonic extraction in an ice bath for 10 min, and centrifuged at 20,000 rpm. The supernatant was transferred to a new EP tube. Then, another extraction solution (methanol:dichloromethane:ethyl acetate=1:1:1) was added to the EP tube containing the mycelial sample for the second extraction. Finally, the two extracts were mixed, centrifuged at 20,000 rpm for 10 min, filtered through a 0.22 um filter membrane into an injection vial and stored in a refrigerator at −20° C. until to be uploaded to a machine.

(3) Mass Spectrometry Analysis

The conditions for mass spectrometry detection were as follows.

Chromatographic separation was performed on a high performance liquid chromatography (Dionex, Sunnyvale, Calif., USA), wherein a chromatographic column is a C₁₈ reverse chromatographic column; mass spectrometry was performed by using an Orbitrap Fusion electrostatic orbitrap high-resolution mass spectrometer (Thermo Scientific, USA), and the liquid chromatographic method uses a mobile phase A: a mixed solution of methanol/water (95/5, v/v, containing 0.1% formic acid and 10 mM ammonium formate) and a mobile phase B: a mixed solution of water/methanol (95/5, v/v, containing 0.1% formic acid and 10 mM ammonium formate). The gradient elution procedure was: 0-1 min: 85% of the phase A, 1-3 min: 85%-50% of the phase A, 3-5 min: 50%-30% of the phase A, 5-10 min: 30%-0% of the phase A, 10-13 min: 0% of the phase A, 15 min: 0-85% of the phase A, 15-20 min: 85% of the phase A (the balance is the phase B). The conditions for the described high-resolution mass spectrometry: ion source heating temperature of 300° C.; spray voltage: 3.5 Kv in a positive ion mode and 3.0 Kv in a negative ion mode; sheath gas of 40 Arb; auxiliary gas of 5 Arb; ion transport capillary temperature of 320° C. and capillary voltage of −1.9 Kv. The main first-order accurate mass number full scan parameters were as follows: Orbitrap was selected as a detector, the resolution was selected as 120,000 FWHM (a half-peak width), the scan range was 100-1,000 m/z, the automatic gain control was set to 1.0e⁶, and the injection time was 100 ms. The main filtering parameters between the primary and secondary scans were as follows: the intensity threshold was 1.0e⁴, the number of charge was 1-2, and dynamic exclusion was set to be 1. A top speed mode was selected for data dependent acquisition, and the cycle time was set to be 1 s. The main secondary mass spectrometry scan (dd-ms²) parameters were as follows: a higher energy collision induced dissociation (HCD) mode was selected as a fragmentation mode, and the collision energy was set to be 35 ev in a positive ion mode and 30 ev in a negative ion mode. The detector type was Orbitrap, the resolution was set to be 30,000 FWHM, and the automatic gain control was set to be 5.0e⁴. The maximum injection time was set to be 100 ms, and the quadrupole isolation width was set to be 1 Da.

(4) Compound Identification

The raw mass spectrogram data were imported into the metabolome data processing software Compound Discovery 2.1, and more than 1483 metabolic features were detected. We manually identified a total of 217 metabolites by comparing the online database with the local database in combination with the relevant literature information of the species we studied. In addition, we sorted all the extracted metabolic features by a peak area size, and the metabolic features not identified in this study were still retained and added to a data matrix for subsequent multivariate statistical analysis. The compounds were characterized by secondary matching scores obtained through comparing the mzCloud database. The local database was also compared.

(5) Screening of Important Warning Molecules

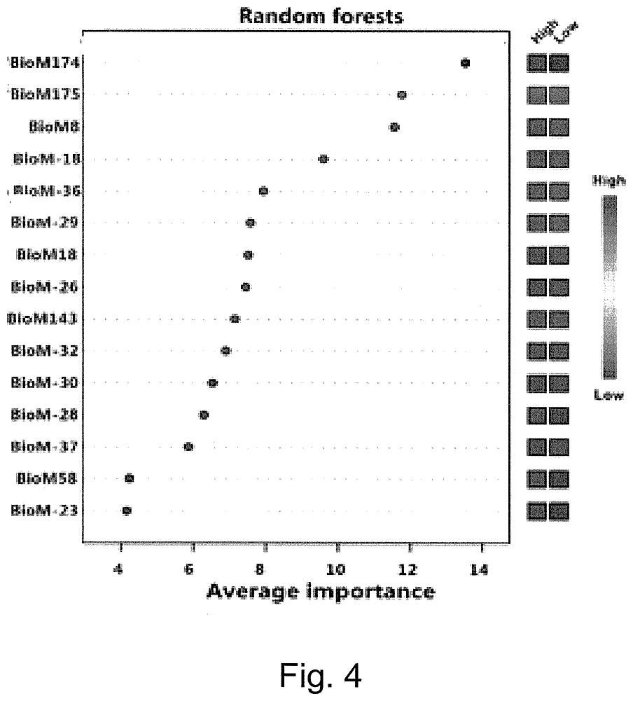

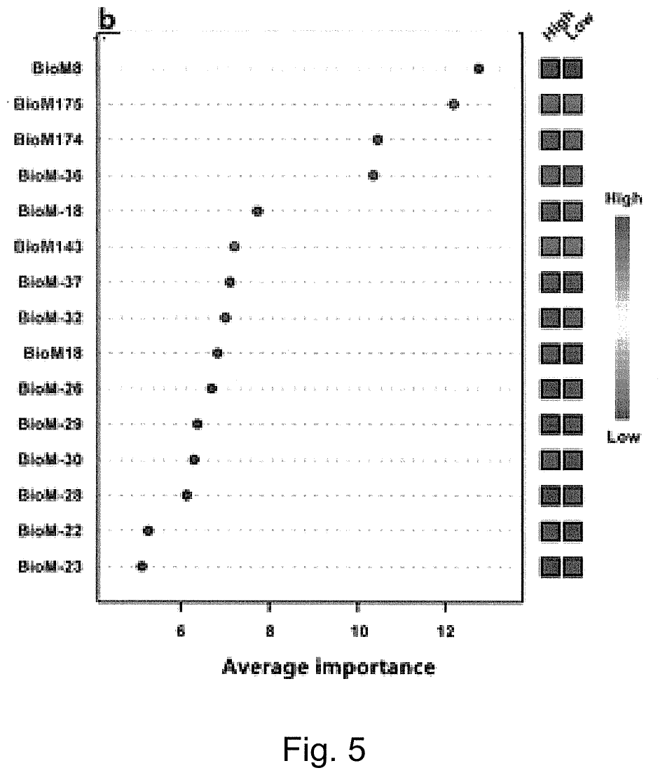

The metabolome dataset from 334 randomly selected above Aspergillus flavus strains was used as a model training set. A large number of candidate differential warning molecules were first screened by using univariate and simple multivariate statistical analysis tools. Based on more than 30 important warning molecules screened above, the importance of each candidate warning molecule to the model was evaluated and ranked in order to screen the most effective warning molecules of toxigenic Aspergillus flavus, and the warning molecules were ranked. FIG. 4 shows the 15 most important warning molecule candidates screened by the model, including the front-ranked BioM8 (5-Methoxysterigmatocystin), BioM-36 (Versicolorin_B), BioM-26 (Versicolorone), and so on. The remaining 258 samples were used as an independent validation set to validate the warning molecules, and the validation results showed that these warning molecules were also screened in the independent validation set and ranked in the front (as shown in FIG. 5). The model constructed by these warning molecules had the best prediction accuracy. Therefore, we chose these warning molecules to construct the warning molecules of the toxigenic Aspergillus flavus strains to effectively differentiate the toxigenic Aspergillus flavus at the subspecies level.

(6) Further Method Validation

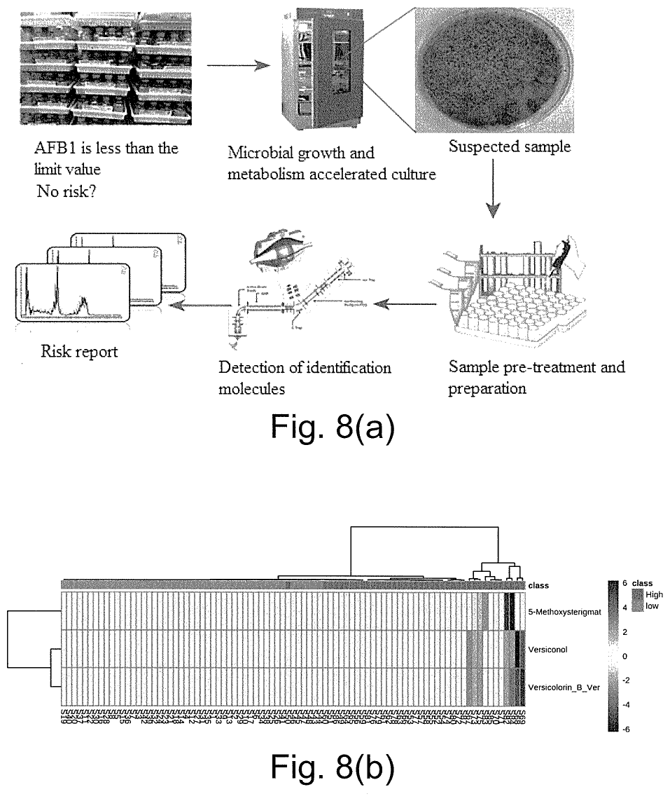

To ensure the reliability and general applicability of the results, methodological corroboration was performed by a detection limit, a quantification limit, precision, linearity and specificity. The detection limit is calculated when the signal-to-noise ratio is greater than 3, and the quantification limit is calculated when the signal-to-noise ratio is greater than 10. The precision of the method was assessed by calculating the measurement error using intra-day continuous injection and inter-day non-continuous 3 days. Linearity range was assessed by weighing 200 mg of mycelia, preparing crushed Aspergillus flavus mycelia, and diluting to make a standard curve with a gradient of 0, 0.01, 0.05, 0.1, 0.5, 1, 2, 5, 10, and 100 μg/mg. The detection limit and quantification limit for aflatoxin and the warning molecules of toxigenic Aspergillus flavus were 0.003-0.10 and 0.012-0.40 μg/mg (mycelia), respectively, with intra-day and inter-day precision of 0.02-0.11 and 0.06-0.12, and R2 of 0.9993-0.9999. See Table 2 below for details. The quantitative standard curves for aflatoxin B1 and toxigenic warning molecules were shown in FIG. 7.

TABLE 2
Methodological corroboration parameters for warning molecules

| | | | Linearity | Detection | Quantification | RSD % | RSD % |
|---|---|---|---|---|---|---|---|
| | Regression | | range | limit | limit | (intra-day | (inter-day |
| warning molecule | equation | R² | (μg/mg) | (μg/mg) | (μg/mg) | precision) | precision) |
| Aflatoxin B₁ | Y = 12044.5X + 1.1*10⁷ | 0.9995 | 0.1-100 | 0.003 | 0.012 | 0.10 | 0.10 |
| 5-Methoxysterigmatocystin | Y = 317.3X − 114190.2 | 0.9999 | 0.5-100 | 0.13 | 0.43 | 0.06 | 0.08 |
| Versicolorin_B | Y = 232.9X − 142191.5 | 0.9993 | 0.1-100 | 0.06 | 0.23 | 0.11 | 0.12 |
| Versiconol | Y = 62.5X + 39562.3 | 0.9998 | 0.1-100 | 0.10 | 0.40 | 0.02 | 0.06 |

RSD: relative standard deviation

The specificity of the biological warning molecules discovered in this study was assessed by analyzing the metabolomes of other fungi isolated from peanut rhizosphere soil and present in agricultural products and comparing the presence or absence of biological warning molecules in other fungi. By comparing 15 other fungi isolated from peanut rhizosphere soil and peanut samples, we found that the warning molecules of toxigenic Aspergillus flavus were not found in other fungi, but only present in the aflatoxigenic Aspergillus parasiticus. This was shown in Table 3 below. This indicated that the warning molecules reported in the present patent may have good specificity for distinguishing toxigenic fungi at the subspecies level. This useful study may be extended to other toxigenic fungi as well.

TABLE 3
Results of specificity assessment of 15 different fungal strains isolated from rhizosphere soil

| Strains | Species | 5-methoxysterigmatocystin | versiconol | versicolorin_B |
|---|---|---|---|---|
| AnHHF-33 | Aspergillus oryzae | − | − | − |
| HeBHD-1 | Aspergillus oryzae | − | − | − |
| HuBLT-3 | Aspergillus oryzae | − | − | − |
| CJ-3-3 | Aspergillus ochraceus | − | − | − |
| BNCC336184 | Aspergillus ochraceus | − | − | − |
| HuBzhx-43 | Aspergillus fumigatus | − | − | − |
| LNCT-4 | Aspergillus parasiticus | + | + | + |
| BNCC340687 | Fusarium moniliforme | − | − | − |
| FJQ2H-4 | Rhizopus oryzae | − | − | − |
| GDHZ-1 | Pichia guilliermondii | − | − | − |
| GXWM-1 | Penicillium janthinellum | − | − | − |
| D83 | Trichoderma spp. | − | − | − |
| XZ-2-9 | Trichoderma spp. | − | − | − |
| XZ-11-5 | Trichoderma spp. | − | − | − |
| BNCC143078 | Fusarium oxysporum | − | − | − |

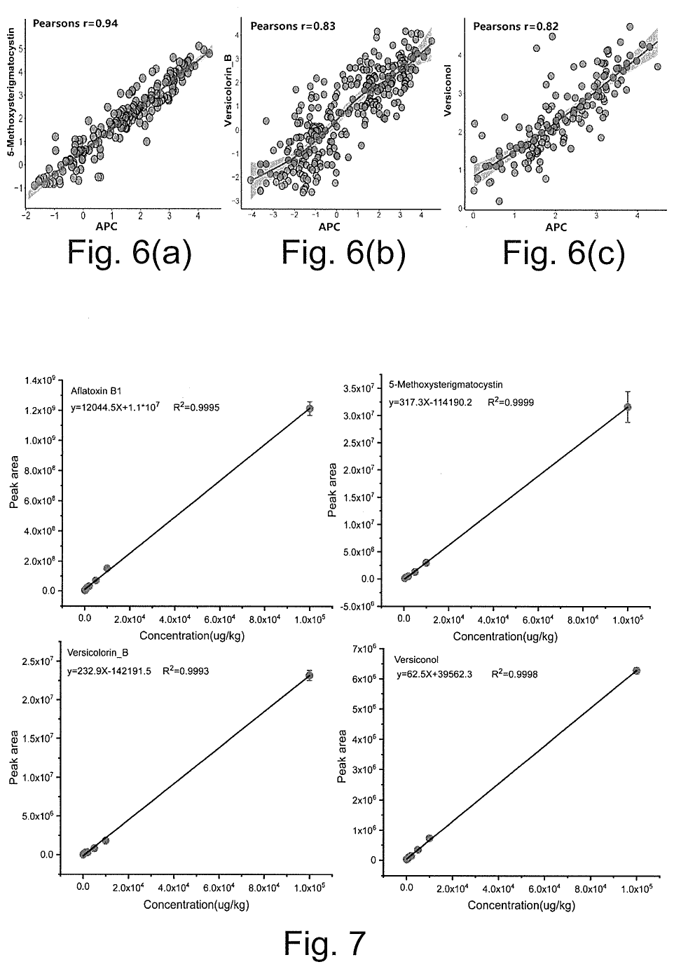

In the present disclosure, the content of 3 molecules described above and the toxigenicity value of Aspergillus flavus were measured, and the data were normalized to take a log value and then subjected to spearman correlation analysis to obtain the following results. The correlations between each warning molecule and the toxigenicity of Aspergillus flavus were 5-methoxysterigmatocystin (5-MST) (r=0.94), versiconol (VOH) (r=0.83), and versicolorin_B (r=0.82), respectively, as shown in FIG. 6(a)-(c).

According to the present disclosure, warning molecules BioM8 (5-Methoxysterigmatocystin), BioM-36 (Versicolorin_B), and BioM-26 (Versicolorone) are screened for the first time by selecting representative Aspergillus flavus population strains on a large scale for metabolomic study and using a combined machine learning analysis approach, and methodological corroboration and specificity assessment are performed on the general applicability of the warning molecules, while practical applications are performed to achieve good early classification and identification effects, providing an original biological warning molecule for early warning of toxigenic aflatoxin in agricultural products. This study uses advanced metabolomics technology combined with advanced machine learning differential screening technology for the development of mycotoxin early warning molecules for the first time, providing a research example for early warning of agricultural product quality and safety in China, with important reference values in theoretical research and practical application.

### Embodiment 2 Early Warning Study of Toxigenic Aspergillus flavus in Agricultural Products

There is a neglected problem in the current risk assessment process of actual peanut samples, that is, usually, whether the aflatoxin in the sample exceeds the standard is only detected, and the potential risk of samples that do not exceed the standard is still poorly understood. It is assumed that if the peanut samples are infested with Aspergillus flavus, humidity and other conditions are not suitable for the growth of Aspergillus flavus, Aspergillus flavus is temporarily in a dormant state, and aflatoxin in the peanut samples is not exceed the standard at this time, or even can not be detected. Once the temperature and humidity conditions are suitable, such samples will face a great risk of aflatoxin contamination.

To solve the above-mentioned problems, we apply the above developed warning molecules of toxigenic Aspergillus flavus. The peanut samples of which the content does not exceed the standard by the detection of the aflatoxin content were added to a mold selection medium and placed in an incubator at 29° C. with a humidity of 90% for incubation. The suspected contaminated samples will grow mold visually. The suspected samples were subjected to sample pre-treatment to extract the warning molecules of toxigenic Aspergillus flavus, and then these warning molecules were analyzed and detected to achieve effective differentiation between contaminated and uncontaminated samples through chemometrics methods.

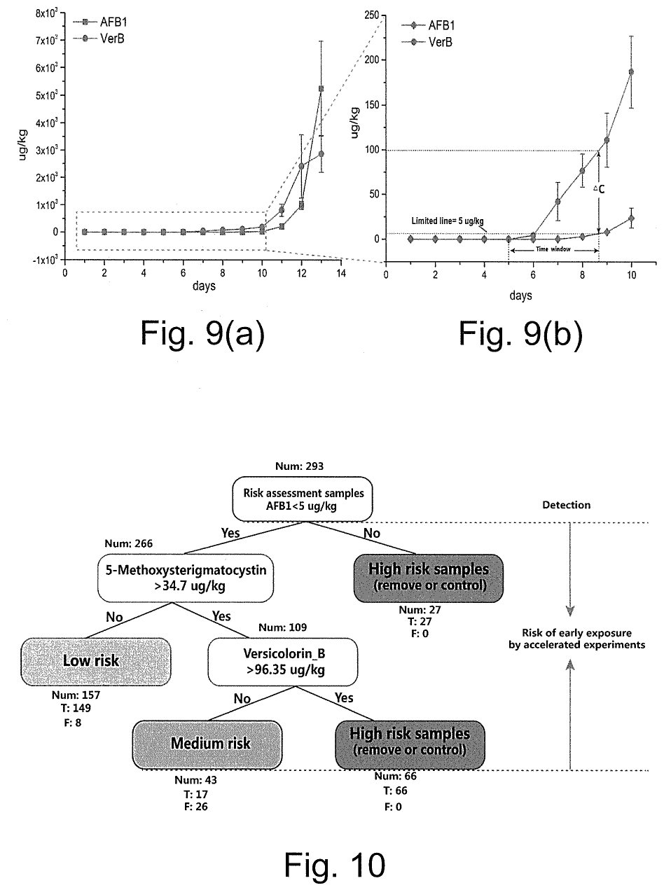

Therefore, according to the present disclosure whether the sample has been contaminated by the toxigenic Aspergillus flavus before the toxin exceeds the standard can be determined in a short time by early detection for warning molecules of the toxigenic Aspergillus flavus. FIG. 8(a) is a recommended workflow for early detection of toxigenic Aspergillus flavus in actual peanut samples. The samples that did not exceed the standard was firstly subjected to microbial growth and metabolism culture, and the suspected samples were screened. The suspected samples were subjected to pre-treatment, warning molecules of toxigenic aspergillus were detected, and then a risk discrimination report was carried out according to classification prediction model or intuitive decision rules. Specifically, in the aflatoxin risk assessment process of actual agricultural products such as peanut, the samples that exceed the standard were screened out according to the detected aflatoxin monitoring value, which are considered to be high-risk samples. Then, the samples that did not exceed the standard or were not detected were subjected to accelerated microbial growth experiments to screen out the suspected samples with Aspergillus growth. For these samples, early detection of warning molecules was carried out in the early stages of culture, and risk assessment and discrimination were performed according to the established early warning model or the developed simple and intuitive decision workflow.

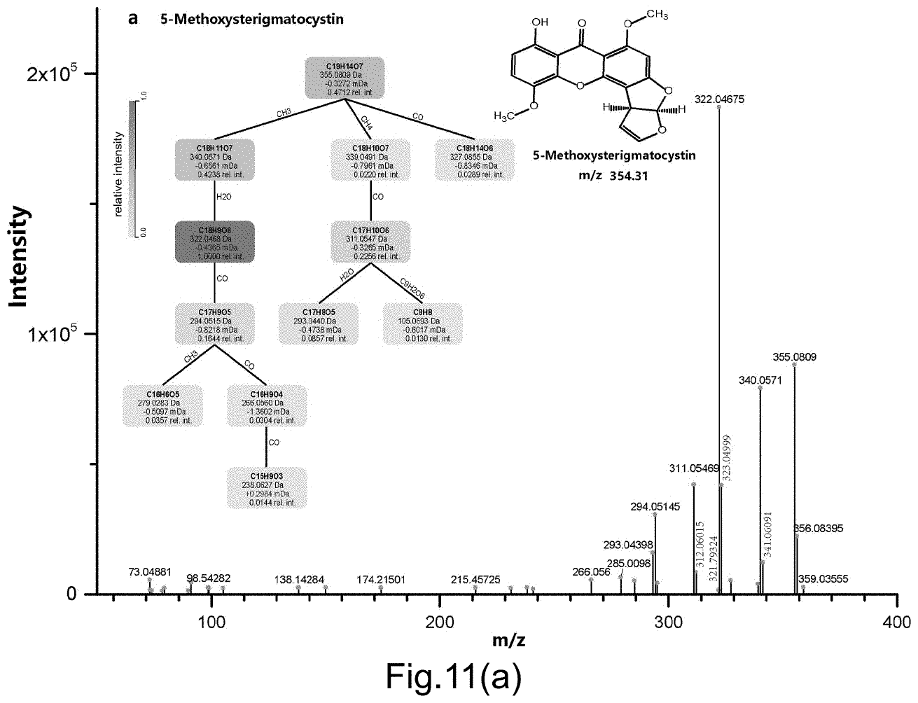

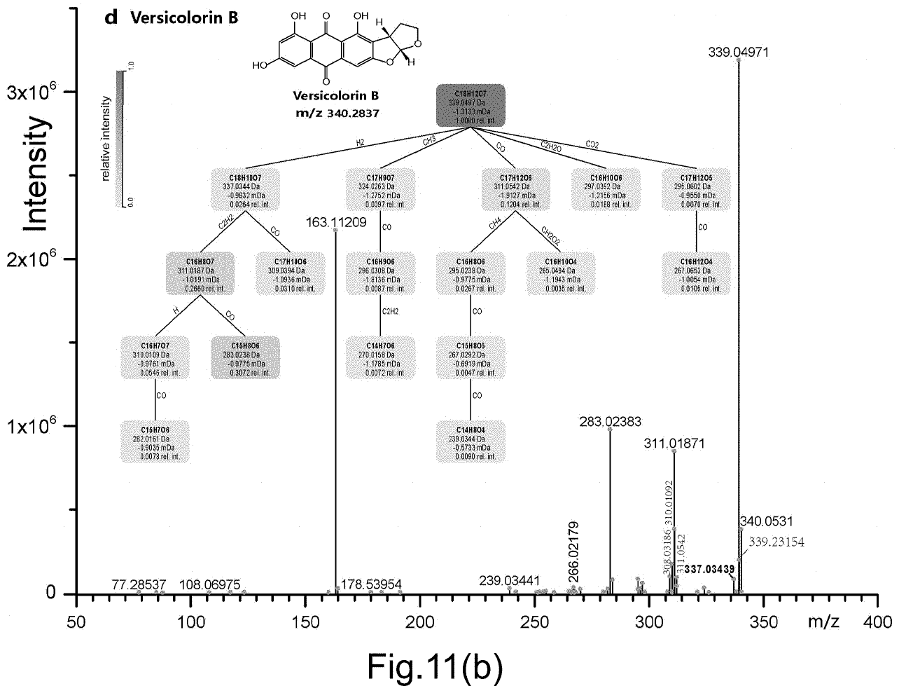

After cultivating and screening 429 samples of peanuts that did not exceed the standard, the samples with mold growth were identified as suspected samples. 86 suspected peanut samples were screened (as shown in FIG. 8(a)), and the warning molecules of toxigenic Aspergillus flavus were detected. The hierarchical clustering analysis in multivariate statistical analysis software such as R language software package can effectively classify the samples contaminated with toxigenic Aspergillus flavus and the samples contaminated with non-aflatoxigenic fungi. 39 samples among these were found to be contaminated with toxigenic Aspergillus flavus, and the results are shown in the heat map of FIG. 8(b). Then, we took one of the samples contaminated with toxingenic Aspergillus flavus for time series culture and found that, as shown in FIG. 9(a)-(b), the results show that the warning molecule VerB related to the severity of the contamination may be detected on the fifth day under the best culture conditions. At this time, the content of aflatoxin was very low. After 3 days, the aflatoxin content exceeded the standard. At this time, the VerB content was 13.8 times the aflatoxin content. The results are shown in FIGS. 9(a) and 9(b). In order to more easily and intuitively determine whether the samples identified as non-exceeding at an early stage were contaminated with toxigenic Aspergillus flavus, we used an advanced interpretable machine learning model, and further collected 180 actual peanut samples as a training set, and developed an operable decision workflow, as shown in FIG. 10. According to the simple and intuitive operational workflow developed, we first classified the samples with and without exceeding the standard according to the results of the risk assessment test, and the samples that exceeded the standard were processed by toxin abatement, or directly destroyed. For the samples that did not exceed the standard, the samples were incubated according to our recommended workflow, and after 3-4 days, samples were taken for detection of the warning molecules of toxigenic Aspergillus flavus, if the content of 5-Methoxysterigmatocystin is greater than a threshold value of 34.7 μg/kg, and whether the content of VerB was greater than 96.35 μg/kg was used to determine the contamination risk of the sample.

The above are only the preferred embodiments of the present disclosure. It should be noted that for those skilled in the art, several improvements and changes can be made without departing from the inventive concept of the present disclosure. For example, the toxigenic Aspergillus flavus described herein may be extended to the category of toxigenic microorganisms, which have similar research ideas to the disclosure, and also belong to the protection scope of the present disclosure.

What is claimed is:

1. Use of an aflatoxin contamination risk warning molecule in aflatoxin contamination risk warning, wherein the aflatoxin contamination risk warning molecule is one or a combination of more than one of versiconol, versicolorin B, and 5-methoxysterigmatocystin.

2. The use according to claim 1, wherein the main secondary mass spectrometry ion peaks of the warning molecule 5-methoxysterigmatocystin (5-MST) C₁₉H₁₄O₇ comprise 350.0809 Da, 340.0571 Da, 322.04675 Da, 311.05469 Da and 285.0098 Da;
the secondary mass spectrometry ion peaks of the warning molecule versiconol (VOH) comprise: 329.06546 Da, 341.09506 Da, and 359.07596 Da; and
the main secondary mass spectrometry ion peaks of the warning molecule versicolorin B (Ver B) comprise: 311.0542 Da, 311.0187 Da, and 283.0238 Da.

3. A method for warning aflatoxin contamination risk based on a warning molecule of an aflatoxigenic strain, comprising the following steps:
weighing a quantitative sample, extracting the aflatoxin contamination risk warning molecule to obtain a sample extract, and detecting and analyzing the sample extract to obtain a quantitative result of the aflatoxin contamination risk warning molecule; and
performing modeling with a chemometrics method by using the content of one or more of the aflatoxin contamination risk warning molecules as a variable to obtain a classification prediction model, inputting the quantitative result of the aflatoxin contamination risk warning molecule, and outputting a risk assessment result based on the classification prediction model to warn aflatoxin contamination of the sample;
wherein the warning molecule of the aflatoxigenic strain is one or a combination of more than one of versiconol (VOH), versicolorin B (Ver B), and 5-methoxysterigmatocystin (5-MST).

4. The method according to claim 3, wherein the chemometrics method is a multivariate statistical analysis method including hierarchical cluster analysis, least partial square orthogonal projection or random forest.

5. The method according to claim 3, wherein after the sample is cultured for 3-4 days, the sample is taken to detect a warning molecule of toxigenic Aspergillus flavus, and a quantitative value of the warning molecule is directly input into the classification prediction model to predict the aflatoxin contamination risk.

6. The method according to claim 3, wherein after the sample is cultured for 3-4 days, the sample is taken to detect a warning molecule of toxigenic Aspergillus flavus, if the content of 5-methoxysterigmatocystin is greater than a threshold value of 34.7 μg/kg, whether the content of VerB is greater than 96.35 μg/kg is further used to determine the contamination risk of the sample, if the content of VerB is greater than 96.35 μg/kg, the sample is a high-risk aflatoxin contamination sample, and if the content of VerB is less than or equal to 96.35 μg/kg, the sample is a medium-risk aflatoxin contamination sample; and then the medium-risk sample is further input into the accurate classification prediction model for verification.

7. The method according to claim 3, further comprising screening a suspected sample, pre-processing the screened suspected sample, detecting the warning molecule of toxigenic Aspergillus, and outputting the risk assessment result based on the classification prediction model to conduct warning assessment of the risk of aflatoxin contamination of the sample, which includes: detecting the aflatoxin content of the sample, and subjecting a sample in which aflatoxin is not detected or the aflatoxin content does not exceed the standard to an accelerated microbial metabolism culture experiment, wherein Aspergillus will grow in the suspected contaminated sample, quenching the suspected sample with liquid nitrogen and grinding for later use, and detecting the aflatoxin content of the sample, wherein a sample with the aflatoxin content higher than a national limit standard is directly identified as a high-risk sample, which is the suspected sample.

8. The method according to claim 3, wherein the sample is an agricultural product or food; extracting the aflatoxin contamination risk warning molecule comprises: performing first extraction by using a solution with a volume ratio of methanol to acetonitrile to water being (2-4):(2-4):(0-1), and then performing second extraction by using a solution with a volume ratio of methanol to dichloromethane to ethyl acetate being (1-3):(1-2):(1-2) to extract the aflatoxin contamination risk warning molecule, and then centrifugating at a high speed to obtain the sample extract.

9. The method according to claim 3, wherein the method for detecting and analyzing the sample extract comprises: subjecting the sample to detection and analysis by liquid chromatography-high resolution mass spectrometer.

10. The method according to claim 3, wherein in the detection and analysis by the liquid chromatography-high resolution mass spectrometer: a chromatographic column is a C₁₈ reverse chromatographic column, and an acquisition mode is divided into a positive ion mode and a negative ion mode which are operated separately, and the acquisition mode is a data-dependent acquisition mode, and primary mass spectrometry data and secondary fragment ion data are simultaneously acquired to perform qualitative and quantitative analysis, thereby obtaining the analysis results of the warning molecule; and the detection and analysis by the liquid chromatography-high resolution mass spectrometer contains an internal standard substance, the internal standard being camphoric acid (the negative ion mode) and 2-chlorophenylalanine (the positive ion mode).

11. The method according to claim 10, wherein the qualitative analysis of the warning molecule comprises: determining a mass deviation within 5 ppm according to the accurate mass number of the primary mass spectrometry of the warning molecule, and then comparing main characteristic ion peaks of the secondary mass spectrometry in combination with the secondary mass spectrogram to perform the qualitative analysis; and the quantitative analysis comprises: in combination with the internal standard substance, performing the quantitative analysis based on a pre-established standard curve of the chromatographic peak area/the peak area of the internal standard-warning molecule concentration.

12. The method according to claim 3, wherein the main secondary mass spectrometry ion peaks of the warning molecule 5-methoxysterigmatocystin (5-MST) C₁₉H₁₄O₇ comprise 350.0809 Da, 340.0571 Da, 322.04675 Da, 311.05469 Da and 285.0098 Da;
the secondary mass spectrometry ion peaks of the warning molecule versiconol (VOH) comprise: 329.06546 Da, 341.09506 Da, and 359.07596 Da; and
the main secondary mass spectrometry ion peaks of the warning molecule versicolorin B (Ver B) comprise: 311.0542 Da, 311.0187 Da, and 283.0238 Da.

13. The method according to claim 6, further comprising screening a suspected sample, pre-processing the screened suspected sample, detecting the warning molecule of toxigenic Aspergillus, and outputting the risk assessment result based on the classification prediction model to conduct warning assessment of the risk of aflatoxin contamination of the sample, which includes: detecting the aflatoxin content of the sample, and subjecting a sample in which aflatoxin is not detected or the aflatoxin content does not exceed the standard to an accelerated microbial metabolism culture experiment, wherein Aspergillus will grow in the suspected contaminated sample, quenching the suspected sample with liquid nitrogen and grinding for later use, and detecting the aflatoxin content of the sample, wherein a sample with the aflatoxin content higher than a national limit standard is directly identified as a high-risk sample, which is the suspected sample.
